(19) United States
(12) Patent Application Publication
(10) Pub. No.: US 2012/0059237 A1
(43) Pub. Date: Mar. 8, 2012

(54) SYSTEM AND METHOD FOR MONITORING BLOOD GLUCOSE LEVELS NON-INVASIVELY

(76) Inventors: Jack Amir, Mazkeret Batya (IL); Itai Arad, Kfar HaOranim (IL); Amir Ben Shalom, Modin (IL); Gusti Aberbuch, Modiin (IL); Tsahi Asher, Kfar HaOranim (IL)

(21) Appl. No.: 13/318,611

(22) PCT Filed: May 4, 2010
(86) PCT No.: PCT/IL10/00353

§ 371 Date: Nov. 3, 2011

Related U.S. Application Data
(60) Provisional application No. 61/175,066, filed on May 4, 2009.

(51) Int. Cl. A61B 5/145

(52) U.S. Cl. 600/365

(57) ABSTRACT

A system and method is described for non-invasive monitoring of blood glucose levels. The system includes a pulse-sensor unit configured to detect a pulse wave travelling through a blood vessel and a processor unit configured to determine pulse wave velocity, to calculate the blood density and so to determine blood glucose level. Various embodiments include pulse-sensor arrays and wearable units configured to communicate with insulin pumps worn about the person of the subject.

### FIELD OF THE INVENTION

The present invention relates to systems and methods for monitoring blood glucose levels. In particular the invention relates to the non-invasive monitoring of blood glucose levels.

### BACKGROUND

Diabetes mellitus is a condition in which blood glucose level of a subject is abnormally high. High blood glucose may be a consequence of a subject's cells not responding to insulin or because of insufficient insulin being produced by the subject's body. As a result, excess glucose accumulates in the blood which may lead to various physiological complications such as vascular, nerve, and other complications. According to the World Health Organization, in the year 2000 approximately 171 million people or 2.8% of the global human population suffered from diabetes. This number is rising annually.

Although all forms of diabetes are treatable, successful treatment depends upon regular monitoring of a subject's blood glucose levels. A blood glucose test may be performed by drawing blood from the subject and testing the sample for glucose content. Typically, samples are collected by piercing the skin of the finger (the pin-prick test).

Continuous blood glucose monitoring (CGM) may be used to determine blood glucose levels at more frequent intervals, typically, every few minutes or so. Invasive techniques are normally used involving the placement of a sensor under the skin which communicates with a receiver configured to display or monitor the readings. It will be appreciated that an invasive sensor may be uncomfortable for the subject. Moreover, an invasive sensor implant typically needs replacing every few days.

It is noted that CGM systems generally monitor glucose levels of interstitial fluid rather than blood glucose levels directly. Thus they typically need to be calibrated regularly with pin-prick tests. Furthermore, the interstitial fluid glucose level tends to lag behind the blood glucose level. Because of this time lag, blood sugar levels may read in the normal range on a CGM system while in reality the patient is already experiencing symptoms of an out-of-range blood glucose value, and treatment may be unduly delayed.

Nevertheless, continuous monitoring allows examination of how the blood glucose levels react to insulin, exercise, food, and other factors. The additional data can be useful for setting correct insulin dosing ratios for food intake and correction of hyperglycemia. Furthermore automatic alerts may be provided for patients at immediate risk of hyperglycemia or hypoglycemia so that corrective action may be taken.

Because of the inconvenience of invasive systems, various non-invasive CGM techniques have been suggested. These include techniques such as near IR detection sensors, ultrasound and dielectric spectroscopy and the like. Non-invasive continuous glucose monitoring may be more convenient to use, however the accuracy and reliability of currently available non-invasive systems is insufficient.

The need remains, therefore, for an effective non invasive continuous blood glucose monitor. The various embodiments described herein address this need.

### SUMMARY OF THE EMBODIMENTS

A first aspect of the embodiments described herein is to disclose a system for non-invasive monitoring of blood glucose levels, the system comprising: at least one pulse-sensor unit configured to detect a pulse wave travelling through a blood vessel and a processor unit configured to receive input from the at least one pulse-sensor unit, to determine pulse wave velocity and to calculate the blood glucose level.

According to some embodiments, the pulse-sensor unit comprises at least two sensors separated by a known spacing distance. Alternatively, the pulse-sensor unit comprises at least one array of sensors for detecting pulse waves. Optionally, the pulse-sensor unit may comprise at least one piezoelectric element. Typically, the piezoelectric element is configured to detect vibrations indicating a pulse wave passing through the blood vessel.

Additionally, the system may further comprise at least one auxiliary sensor. The at least one auxiliary sensor may be configured to monitor the subject. Alternatively, the at least one auxiliary sensor may be configured to monitor internal parameters of the system. For example, the one auxiliary sensor may comprise an oximeter. In another example, the auxiliary sensor may comprise a sensor-temperature monitor configured to monitor operating temperature of at least one pulse-sensor. Typically, the processor is configured to receive additional signals from the auxiliary sensors. Advantageously, the processor is configured to adjust the blood glucose level calculation according to input from at least one auxiliary sensor.

Optionally, where the pulse-sensor unit comprises at least one array of sensors, each sensor of the array is configured to collect a set of pressure samples detected at short time intervals δt. Accordingly, the processor may be configured to select a first set of pressure samples from a first sensor and a second set of pressure samples from a second sensor. The processor may be further configured to measure the degree of correlation between the first set of pressure samples to the second set of pressure samples at a plurality of time shifts τ. Preferably, the processor is further configured to select the time shift τₒₚₜ with highest degree of correlation. Generally, the pulse wave velocity is determined by dividing the distance between the first sensor and the second sensor by the optimal time shift τₒₚₜ.

The system may be incorporated into a standalone unit. Alternatively, the system may comprise a satellite unit in communication with a base unit. Preferably, the system comprises a comfortable wearable unit. For example the pulse-sensing unit may be incorporated into a wristband.

Typically, the system further comprises at least one output unit. The output unit may be selected from a group consisting of: display screens, computer memory units, data transmitters, data bases, hard discs, flash memory devices, SD cards, USB ports and the like.

In a particular embodiment, the system further comprises an insulin pump configured to administer at least one dose of insulin to the subject wherein the processor is further configured to calculate the parameters of the dose. For example such parameters may be selected from size, shape and frequency.

According to another aspect embodiments described herein teach a method for monitoring blood glucose levels. The method comprising the steps: producing a calibration curve by measuring pulse wave velocity in blood in a plurality of samples of blood having different glucose levels; measuring the pulse wave velocity in a blood vessel, and comparing the measured pulse wave velocity of the subject with the calibration curve thereby determining the blood glucose level in the subject.

Optionally, the step of measuring the pulse wave velocity in a blood vessel comprises: providing at least one array of sensors; the each sensors of collecting a set of pressure samples detected at short time intervals δt; selecting a first set of pressure samples from a first sensor and a second set of pressure samples from a second sensor; measuring the degree of correlation between the first set of pressure samples to the second set of pressure samples at a plurality of time shifts τ; selecting the time shift τₒₚₜ with highest degree of correlation; determining inter-sensor spacing between the first sensor and the second sensor; and dividing the inter-sensor spacing by the optimal time shift τₒₚₜ.

Another aspect of the embodiments is to teach a general method for measuring the pulse wave velocity comprising: providing at least one array of sensors; each the sensors of collecting a set of pressure samples detected at short time intervals δt; selecting a first set of pressure samples from a first sensor and a second set of pressure samples from a second sensor; measuring the degree of correlation between the first set of pressure samples to the second set of pressure samples at a plurality of time shifts τ; selecting the time shift τₒₚₜ with highest degree of correlation; determining inter-sensor spacing between the first sensor and the second sensor; and dividing the inter-sensor spacing by the optimal time shift τₒₚₜ.

### BRIEF DESCRIPTION OF THE FIGURES

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments of the present invention only, and are presented in the cause of providing what is believed to be the most useful and readily understood description of the principles and conceptual aspects of embodiments of the invention. In this regard, no attempt is made to show structural details in more detail than is necessary for a fundamental understanding of the embodiments; the description taken with the drawings making apparent to those skilled in the art how the several forms of the invention may be embodied in practice. In the accompanying drawings:

FIG. 1 is a block diagram schematically representing the main components of an embodiment of a non-invasive continuous blood-glucose monitor;

FIG. 2 is a schematic representation of a second embodiment of the non-invasive glucose monitor configured to communicate with an insulin pump to automatically regulate blood glucose level;

FIG. 3 is a schematic representation of a third embodiment of the non-invasive glucose monitor incorporated into a wrist band;

FIG. 4 is a block diagram representing the main components of a fourth embodiment of the non-invasive glucose monitor;

FIG. 5 is a flowchart representing a method for measuring pulse wave velocity which may be used in embodiments of the non-invasive blood-glucose monitor.

### DESCRIPTION OF SELECTED EMBODIMENTS

Embodiments of the non-invasive blood glucose monitor disclosed herein utilize the Pulse Wave Velocity (PWV) within a blood vessel as a measure of blood glucose level. In particular it is noted that the higher the glucose level in the blood, the denser it becomes. Because of the increase in the blood's density, for a given blood pressure, the pulse wave speed is slower in blood containing higher levels of glucose.

Pulse Wave Velocity (PWV) is a measure of the speed at which the pulse wave propagates through a blood vessel. PWV is often used medically as an indication of arterial wall stiffness which is associated with the risk of cardiovascular events.

Various factors determining the value of PWV are related according to the Moens-Korteweg equation. The Moens-Korteweg equation states that the pulse wave velocity may be given by:

$$PWV = \sqrt{\frac{Eh}{2 rp}}$$

where E is the incremental Young's modulus of the wall of the blood vessel, h is the thickness of the wall of the blood vessel, r is the radius of the blood vessel and ρ is the blood density.

The Moens-Korteweg equation is typically used to determine the incremental Young's modulus of the wall of the blood vessel from the measured PWV and thereby to monitor arterial wall stiffness.

In contradistinction to normal practice, it is a particular feature of embodiments described herein that the Moens-Korteweg equation may be used to determine the density of the blood and thereby to indicate blood glucose level. It is noted that known blood glucose level monitors of the prior art such as those described above do not use PWV measurements.

By monitoring pulse waves passing multiple locations separated by a known distance along a blood vessel, the PWV and therefore the blood glucose level may be monitored near continuously in real time. It is noted that in order to verify the systems integrity and accuracy, blood glucose levels measured by the system may be periodically calibrated against conventional invasive blood glucose monitoring methods, such as but not limited to the pin prick test.

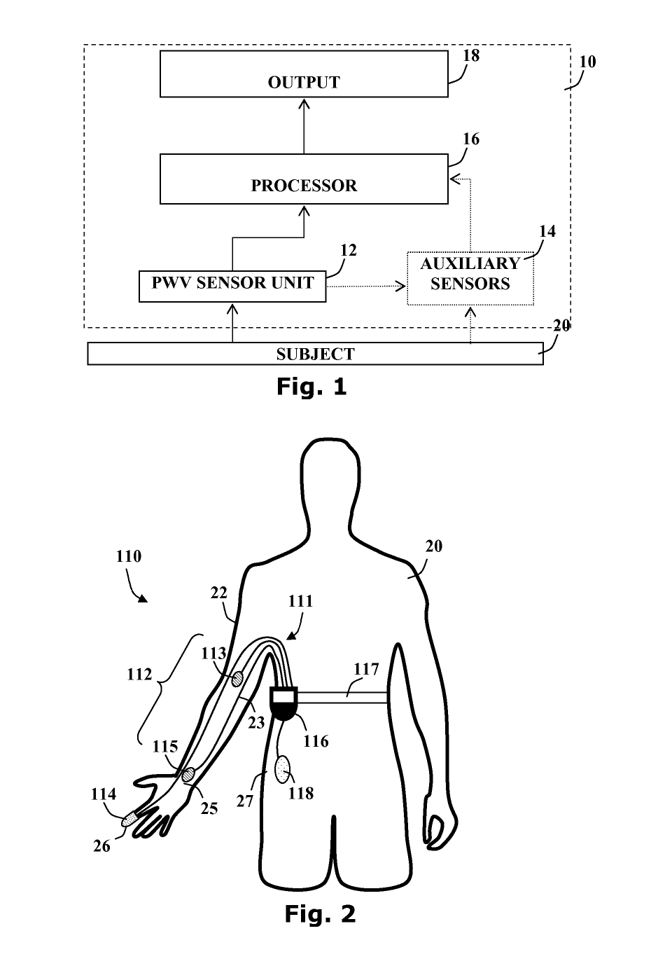

Reference is now made to the block diagram of FIG. 1 schematically representing the main components of one embodiment of a non-invasive continuous blood-glucose monitor 10. The monitor 10 includes a pulse-sensor unit 12, a processor 16 and an output 18.

The pulse-sensor unit 12 is configured to detect and sample a pulse wave typically travelling through a blood vessel (not shown) of a subject 20. The processor unit 16 is configured to receive input from the pulse-sensor unit 12, to determine the pulse wave velocity (PWV), as described in detail below, and thereby to calculate the blood glucose level of the subject 20.

According to some embodiments of the monitor, auxiliary sensors 14 may be included to provide additional data to the processor 16. The processor 16 may use the additional data to adjust its calculations so as to more accurately determine the blood glucose level of the subject 20. Variously, such additional data may relate to the subject 20, for example oxygen content of the blood measured by an oximeter or the like. Alternatively, the data may relate to the internal parameters of the monitor 10 such as data obtained from internal thermometers monitoring the temperature of pulse sensors. Thus, measured values may account for temperature related drift or the like in the sensors. Alternatively, or additionally, humidity detectors, ambient pressure sensors or the like may be configured to monitor other parameters of operational.

The output 18 may be used to display data such as the blood glucose level, pulse wave velocity, pulse wave profile, pulse frequency, blood pressure or the like. Advantageously, the output 18 may further provide alerts, for example audio or visual indications when the monitored parameters fall outside predefined normal ranges.

Other outputs 18 may include data handling devices such as computer memory units, data bases, hard discs, flash memory devices, SD cards, data transmitters, USB ports or the like which may be used to store data for future reference or analysis.

Although only a single block is represented for the monitor 10 in FIG. 1, it will be appreciated that alternatively, the processor 16, output 18 and sensor unit 12 may each be incorporated into separate units.

Referring now to FIG. 2, a schematic representation is shown of a second embodiment of the non-invasive glucose monitor 110 worn by a subject 20. The second embodiment of the monitor 110 includes a central unit 116, a satellite pulse sensor unit 112 and an oximeter 114. The central unit 116 is preferably lightweight and suitable for being worn about a subject's body, for example on the waist on a belt strap 117 or the like.

It is a particular feature of the second embodiment that the monitor 110 is in communication with an insulin pump 118. Such a system may be used to automatically regulate blood glucose level.

The sensor unit 112 of the glucose monitor 110 includes at least two sensors 113, 115 placed at the crook of the elbow 23 and the wrist 25 respectively so as to monitor the pulse wave at two separate locations. The sensors 113, 115 are connected via wires 111 to the central unit 116 housing the processor (not shown). Although a wired connection is described herein in other embodiments alternative data communication methods may be preferred including wireless protocols such as wifi, Bluetooth and the like.

Such sensors may be for example piezoelectric crystal elements which generate a small electric potential when stressed by a passing pressure wave. By rapidly sampling the potential generated across the piezoelectric elements, the vibrations associated with a pulse wave passing through a blood vessel may be detected and furthermore the pulse wave profile may be modeled.

In addition to the pulse sensor unit 112, an auxiliary oximeter sensor 114 is provided. The pulse oximeter 114 may be attached to an extremity such as a finger 26 for example. It is noted that, by analyzing the pulse wave shape the relative blood pressure may be calculated, as well as the oxygen level and heart rate. Accordingly, the system may use blood pressure measurements as a factor in the calculation of blood glucose levels. Furthermore, apart from monitoring oxygen level, the optical signals of the oximeter may help in verification and adjustment of the mechanical signals measured by piezoelectric pulse sensors.

The insulin pump 118 may be attached to the hip 27 of the subject 20 and is configured to administer bolus or basal rate doses of insulin to the subject. It is a feature of the embodiment that the processor may be configured to calculate the size, shape and frequency of the dose to be administered so as to provide real time automatic regulation of the blood-glucose levels of the subject 20. Various insulin pumps 118 are known in the art, and may be used in combination with embodiments of the blood glucose monitors.

Where the sensors are placed at a known separation distance, the pulse wave velocity may be determined by simple arithmetic division of the sensor separation by the time delay between pulse detection at each sensor. It is noted that determination of both the exact sensor spacing is challenging as the sensors may move relative to one another. Furthermore the exact time delay is also known to be difficult to determine because of reflections and interference of pulse waves within the blood vessel. Various systems and methods may be used for successful PWV measurement such as measuring the time of the leading edge of the pulse wave, the use of curve and envelope areas integration or such like calculation means.

It is further noted that, although the pulse sensors 113, 115 of the second embodiment are placed at the crook of the elbow 23 and the wrist 25 where it is relatively easy to sense the pulse wave propagating through the arterial blood vessels of the arm, precise placement of the sensors may be difficult. In order to provide continuous monitoring of the blood glucose level, both of the sensors 113, 115 must be positioned where they can continuously detect the pulse wave. This may be difficult to achieve particularly where a subject is actively moving his limbs.

Alternatively, according to other embodiments, pulse sensors may include an array of pickup sensors. This may make sensor positioning significantly easier as placement of the array over the extended region would typically allow at least one of the pickup detectors to monitor the pulse. Detection data may then be gathered from the selected pickup sensor having optimal readings at the time of the measurement.

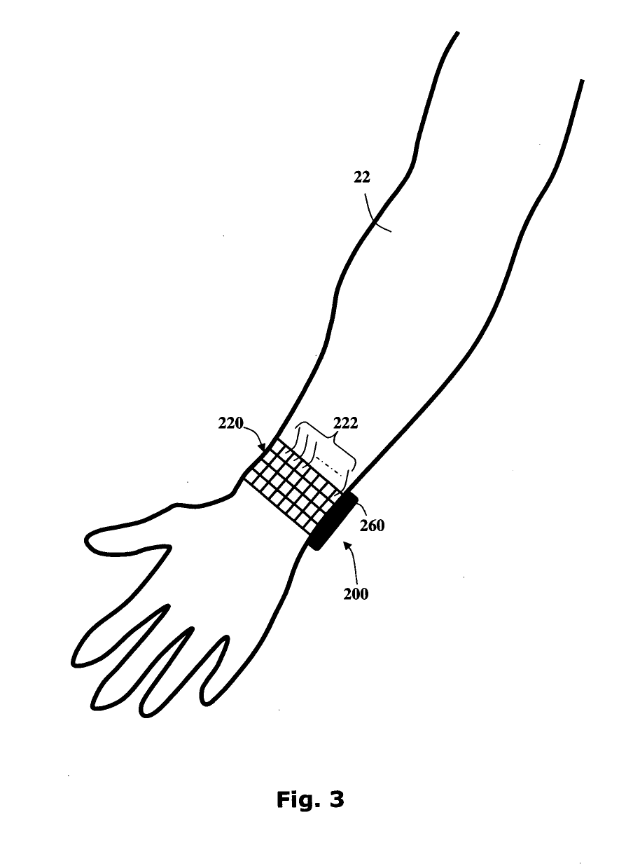

Reference is now made to FIG. 3 showing a schematic representation of a third embodiment of the non-invasive glucose monitor 200 incorporated into a wrist band 220. The monitor 200 includes an array of pickup sensors 222 and a control unit 260. The pickup sensors 222 are embedded in a wrist band 220 such as a piece of elastic material worn tightly around the wrist. At least a portion of the pickup sensors 222 are able to sense the pulse wave passing through the blood vessel. It is noted that the control unit 260 which includes the processor unit (not shown) and other electronic components may be conveniently supported by the wrist band 220. The embodiment of the non-invasive monitor 200 may thus be readily incorporated into an external device, such as a watch and watch strap for example.

According to the third embodiment of the monitor 200, each sensor 222 is configured to sample data at regular time intervals δt. When a pulse wave passes the wristband 222 each of the pickup sensors 222 will typically detect the pulse wave as a set of data samples. Because all the pickup sensors 222 within the array are within close proximity to one another, it will be appreciated that the profile of the pulse wave detected by each pickup sensor 222 will typically be similar. It is noted, however, that each pickup sensor will typically detect the pulse wave at a slightly different time as it passes. Thus, a pulse wave passing the wristband 220 may be detected by two pickup sensors 222 as two similar sets of data samples offset by a time shift τ.

It is a particular feature of the third embodiment of the monitor 200 that the pulse wave velocity may be determined by selecting two sets of data samples from two pickup sensors 222 at a known spacing distance and finding the optimal time shift τₒₚₜ at which the correlation between the two sets of data samples have the highest degree of correlation. The pulse wave velocity may then be calculated as the ratio of spacing distance to optimal time shift τₒₚₜ,

The correlation between a first set of data samples S_{A}={A₁, A₂, A₃ . . . } collected by a first pickup sensor and a second set of data samples S_{B}={B₁, B₂, B₃ . . . } collected by a second pickup sensor may be determined using, for example, the Pearson Product-Moment Correlation Coefficient r_{AB}, where:

$${r}_{AB} = \frac{{\sum }_{i = 1}^{n} ( {A}_{i} - \overline{A} ) ( {B}_{i} - \overline{B} )}{\sqrt{{\sum }_{i = 1}^{n} {( {A}_{i} - \overline{A} )}^{2}} \sqrt{{\sum }_{i = 1}^{n} {( {B}_{i} - \overline{B} )}^{2}}}$$

The optimal time shift τₒₚₜ may be determined by defining a set S_{B′}(τ)={B_{1+τ}, B_{2+τ}, B_{3+τ}, . . . }, temporally shifted from S_{B} by τ, and finding the value of τ with the highest Pearson Product-Moment Correlation Coefficient r_{AB′} by maximizing the function:

$${r}_{{AB}^{′}} ( τ ) = \frac{{\sum }_{i = 1}^{n} ( {A}_{i} - \overline{A} ) ( {B}_{i + τ} - \overline{B} )}{\sqrt{{\sum }_{i = 1}^{n} {( {A}_{i} - \overline{A} )}^{2}} \sqrt{{\sum }_{i = 1}^{n} {( {B}_{i + τ} - \overline{B} )}^{2}}}$$

Although only the Pearson Product-Moment Correlation Coefficient is discussed above, other correlation methods may alternatively be used to determine the optimal time shift τₒₚₜ.

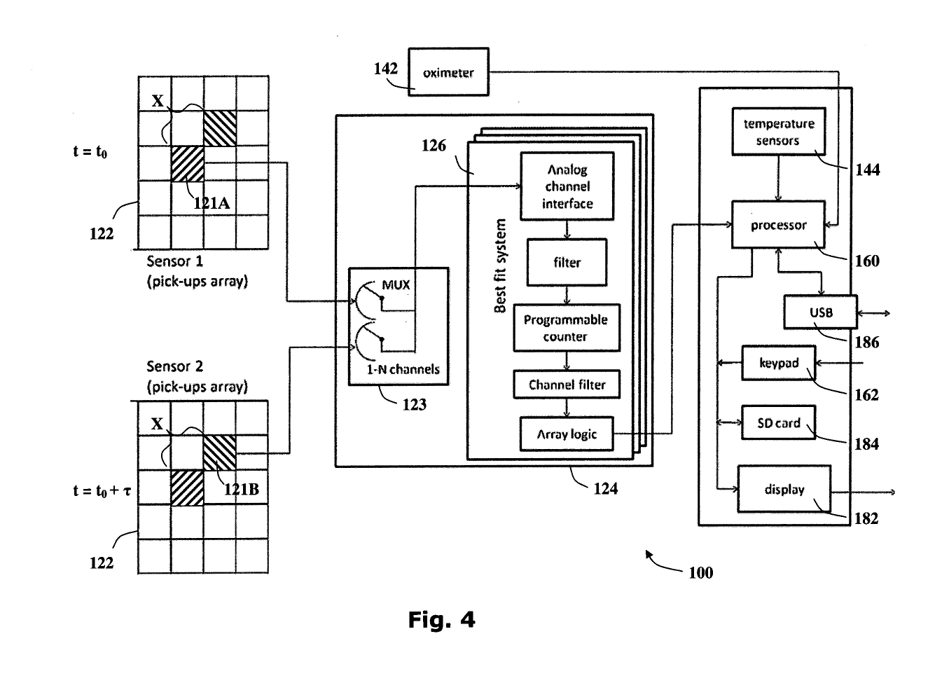

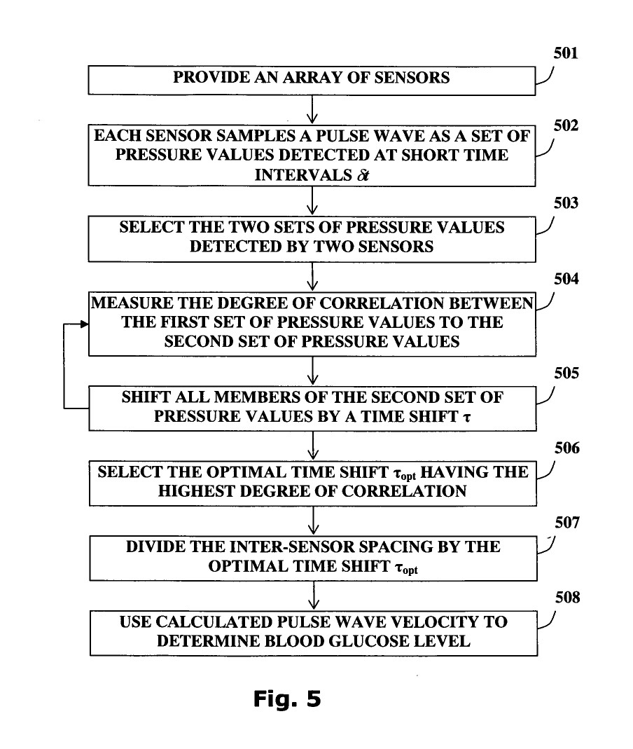

Reference is now made to the block diagram of FIG. 4 representing a fourth embodiment of the non-invasive glucose monitor 100. The fourth embodiment of the monitor 300 includes a sensor unit 120, an oximeter 142, and a processor unit 160.

The sensor unit 120 includes an array 122 of signal pickups 121 and an analyzer 124. Signals from the pickups 121 are analyzed by the analyzer 124 in order to select the two pixels 121A, 121B which produce the best two signal sets from the array 122 according to criteria such as signal-to-noise ratio, amplitude, repetitiveness and signal-stability.

Sets of signals from the selected pickups 121A, 121B may be selected by the MUX 123 and fed into the best fit block 126 of the analyzer 124 as an algorithmic signal input. The channel integrity is monitored throughout the analyzing cycle. The relevant channel position may be fed into the system and the processor 160 is configured to calculate a value the optimal time shift τₒₚₜ for example using a pulse wave correlation method such as described herein. The distance X between the two selected pickups 121A, 121B is then used for performing the PWV calculation.

Typically, the processor unit 160 is configured to calculate the time shift regularly, for example every minute or so and to receive other data for example from an oximeter 142 or other auxiliary monitors such as an internal temperature monitor 144. The signals received by the processor unit 160 are used to calculate the blood glucose levels. Accordingly, sugar level results may be displayed on a user interface 182 and/or recorded on storage medium 184 such as a hard disc, Flash memory, SD card or the like. The signal may also be directed to a USB port 186 for further external storage or analysis. It is further noted that a user input device such as a keyboard 162, touch pad or the like may be further provided.

Other embodiments of the system include standalone machines configured to measure the pulse wave speed over a tested organ to check malfunctions in the blood circulation and external units for communicating with a base station such as a computer or the like by wired or unwired means.

Reference is now made to the flowchart of FIG. 5 illustrating the steps of a method for measuring pulse wave velocity and thereby to determine blood glucose level. The method includes the steps of: providing at least one array of sensors 501; each sensor collecting a set of pressure samples detected at short time intervals δt 502; selecting a first set of pressure samples from a first sensor and a second set of pressure samples from a second sensor 503; measuring the degree of correlation between the first set of pressure samples to the second set of pressure samples 504; shifting the values of second set by a time shift τ 505; repeating previous steps a plurality of times and selecting the time shift τₒₚₜ with highest degree of correlation 506; dividing the inter-sensor spacing by the optimal time shift τₒₚₜ to obtain the pulse wave velocity 507, and using the pulse wave velocity to determine the blood glucose level 508.

It is noted that the step of determining the blood glucose level 508 may include the substeps of producing a calibration curve or a look up table relating PWV to blood glucose level, perhaps by measuring pulse wave velocity in blood in a plurality of samples of blood having different glucose levels; and comparing the measured pulse wave velocity with the calibration curve.

Although the method of calculation of pulse wave velocity is described hereinabove in relation to a blood glucose monitor, it will be appreciated that the method may be applied to other procedures in which the pulse wave velocity determination is required. Thus the embodiments described hereinabove disclose various systems and methods which may be used to measure pulse wave velocity in general and for application in non-invasive blood glucose monitors in particular.

The scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1-28. (canceled)

29. A system for non-invasive monitoring of blood glucose levels, the system comprising: at least one pulse-sensor unit configured to detect a pulse wave travelling through a blood vessel and a processor unit configured to receive input from said at least one pulse-sensor unit, to determine pulse wave velocity and to calculate the blood glucose level wherein said pulse-sensor unit comprises at least two sensors separated by a known spacing distance.

30. The system of claim 29 wherein said pulse-sensor unit comprises at least one array of sensors and each sensor of said array is configured to collect a set of pressure samples detected at short time intervals δt.

31. The system of claim 29 wherein said processor is configured to select a first set of pressure samples from a first sensor and a second set of pressure samples from a second sensor.

32. The system of claim 31 where said processor is further configured to measure the degree of correlation between said first set of pressure samples to said second set of pressure samples at a plurality of time shifts τ.

33. The system of claim 32 wherein said processor is further configured to select a time shift τₒₚₜ with highest degree of correlation and the pulse wave velocity is determined by dividing distance between said first sensor and said second sensor by the optimal time shift τₒₚₜ.

34. The system of claim 29 wherein said pulse-sensor unit comprises at least one piezoelectric element.

35. The system of claim 34 wherein said at least one piezoelectric element is configured to detect vibrations indicating a pulse wave passing through said blood vessel.

36. The system of claim 29 comprising at least one auxiliary sensor.

37. The system of claim 29 comprising at least one auxiliary sensor configured to monitor said subject.

38. The system of claim 29 comprising at least one auxiliary sensor configured to monitor internal parameters of the system.

39. The system of claim 29 comprising at least one auxiliary sensor comprising an oximeter.

40. The system of claim 29 comprising at least one auxiliary sensor comprising a sensor-temperature monitor configured to monitor operating temperature of at least one pulse-sensor.

41. The system of claim 29 wherein said processor is configured to adjust a blood glucose level calculation according to input from at least one auxiliary sensor.

42. The system of claim 29 incorporated into a stand-alone unit.

43. The system of claim 29 comprising a satellite unit in communication with a base unit.

44. The system of claim 29 comprising a comfortable wearable unit.

45. The system of claim 29 wherein said pulse-sensing unit is incorporated into a wristband.

46. The system of claim 29 further comprising at least one output unit.

47. The system of claim 46 wherein said output unit is selected from a group consisting of: display screens, computer memory units, data transmitters, data bases, hard discs, flash memory devices, SD cards and USB ports.

48. The system of claim 29 further comprising an insulin pump configured to administer at least one dose of insulin to said subject wherein said processor is further configured to calculate the parameters of said dose.

49. The system of claim 48 wherein said parameters are selected from size, shape and frequency.

50. A method for monitoring blood glucose levels comprising the steps:
producing a calibration curve by measuring pulse wave velocity in blood in a plurality of samples of blood having different glucose levels;
measuring the pulse wave velocity in a blood vessel, and
comparing the measured pulse wave velocity of said subject with said calibration curve thereby determining the blood glucose level in said subject.

51. The method of claim 50 wherein said measuring the pulse wave velocity in a blood vessel comprises:
providing at least two sensors;
collecting a set of pressure samples detected at short time intervals δt;
selecting a first set of pressure samples from a first sensor and a second set of pressure samples from a second sensor;
measuring the degree of correlation between said first set of pressure samples to said second set of pressure samples at a plurality of time shifts τ;
selecting a time shift τₒₚₜ with highest degree of correlation;
determining inter-sensor spacing between said first sensor and said second sensor; and
dividing the inter-sensor spacing by the optimal time shift τₒₚₜ.

52. A method for measuring the pulse wave velocity comprising:
providing at least two sensors;
collecting a set of pressure samples detected at short time intervals δt;
selecting a first set of pressure samples from a first sensor and a second set of pressure samples from a second sensor;
measuring the degree of correlation between said first set of pressure samples to said second set of pressure samples at a plurality of time shifts τ;
selecting the time shift τₒₚₜ with highest degree of correlation;
determining inter-sensor spacing between said first sensor and said second sensor; and
dividing the inter-sensor spacing by the optimal time shift τₒₚₜ.
